(19) United States
(12) Patent Application Publication
(10) Pub. No.: US 2015/0230944 A1
(43) Pub. Date: Aug. 20, 2015

(54) METHOD FOR ADJUSTING A PRESSURE

(71) Applicant: OTTO BOCK HEALTHCARE GMBH, Duderstadt (DE)

(72) Inventors: Luder Mosler, Duderstadt (DE); Mark Schonemeier, Gottingen (DE)

(73) Assignee: OTTO BOCK HEALTHCARE GMBH, Duderstadt (DE)

(21) Appl. No.: 14/421,816

(22) PCT Filed: Aug. 8, 2013
(86) PCT No.: PCT/EP2013/002371

§ 371 Date: Feb. 14, 2015

(30) Foreign Application Priority Data
Aug. 16, 2012 (DE) 10 2012 016 197.2

(51) Int. Cl. A61F 2/80

(52) U.S. Cl. CPC A61F 2/80; A61F 2002/802

(57) ABSTRACT

A system and method to adjust a pressure in a volume in a negative pressure shaft system. The method includes: a) determining pressure values that constitute a measurement for the pressure in the volume at different points in time, and storing the pressure values that have been determined, b) determining a maximum value and a minimum value from the stored pressure values, c) determining a difference value from the maximum value and the minimum value, d) comparing said difference value with a predetermined first limit value, and e) activating a pump in order to aspirate a fluid from the volume if said difference value is greater than the predetermined first limit value.

The invention relates to a method for adjusting a pressure in a volume in a negative-pressure socket system.

To ensure that prosthesis systems intended to replace amputated limbs can be securely fastened on the amputation stump of the patient, a number of different possibilities are known from the prior art. For example, a liner can first of all be pulled over the amputation stump, said liner being made, for example, of an elastic material such as silicone. This liner ensures a comfortable feel while the prosthesis is being worn and, on account of its elasticity, is able to adopt the optimal shape to fit the respective amputation stump. The actual prosthesis socket, on which the further prosthetic set-up is fitted, must then be arranged on this liner. This is often done using negative pressure, by which the prosthesis system is held on the amputation stump of the patient. The strength of the negative pressure that is present in this volume is critical both in respect of the prosthesis being securely fastened on the amputation stump and also in respect of the prosthesis feeling comfortable for the patient to wear. It is therefore of the greatest importance that this negative pressure, or the pressure present in the volume, can be adjusted as optimally as possible.

If the prosthesis is held on the amputation stump of the patient by a negative pressure, this is referred to as a negative-pressure socket system. The negative pressure is often generated between the liner, pulled over the amputation stump, and a prosthesis socket. However, it is also alternatively conceivable, for example, that the negative pressure is generated directly between a prosthesis socket, or another component of the prosthesis system, and the bare amputation stump. For this purpose, the volume naturally has to be closed off in an airtight manner, which can be done, for example, by a sealing lip which is arranged at the upper end of the prosthesis socket and which is made of a sealing material, for example silicone. In addition to these two very common ways of fastening a prosthesis on the amputation stump of a patient by a negative pressure, a great many other possibilities are also conceivable.

If the negative pressure is set too high, the pressure in the volume is then too low, so that, although a secure fastening of the prosthesis on the amputation stump is ensured, the prosthesis is uncomfortable or even painful for the patient to wear, especially after long periods of being seated or after other extended periods in which the prosthesis is unloaded. By contrast, if too low a negative pressure is generated in the volume, the pressure in the volume is then too great, and the patient often has the sensation that the prosthesis is loose or unstable. Therefore, in modern prosthesis systems, vacuum pumps are often already provided which, by way of a fluid connection, are connected for example to an interior of the prosthesis socket, such that, after the prosthesis system has been fitted in place, the volume between the liner and the prosthesis system can be subjected to a negative pressure by the vacuum pump.

During use of the prosthesis, for example when walking, tension and compression phases occur at each step and, in particular, place a load on the connection between the amputation stump, or the liner pulled over the latter, and the prosthesis socket. The volume between the amputation stump, or the liner, and the prosthesis socket is generally unstable since it comprises layers of tissue and elastic materials for example, such that there may be compressible intermediate volumes arranged between both layers or even partial detachment of the liner from the socket. The tensile and compressive forces exerted by the use of the prosthesis therefore lead to slight fluctuations of the volume in the negative-pressure socket system. Since this volume is closed off hermetically in the optimal case, this leads to pressure fluctuations in the interior of the volume.

A number of ways of controlling the pressure in the volume are known from the prior art.

For example, US 2006/0212128 A1 discloses the provision of an upper limit value and lower limit value for the negative pressure. The pressure difference between the pressure in the volume and an atmospheric pressure surrounding the prosthesis system is measured in each case. This pressure difference has to lie between the two fixed limit values. If the pressure difference is too low, which can occur as a result of slight leakages for example, the pump is activated until the pressure difference lies at the upper limit value. Similar solutions are also proposed in U.S. Pat. No. 8,007,543 B2, US 2011/0060421 A1 and US 2010/0312361 A1.

However, leg prostheses in particular are subject to considerable pressure fluctuations during use since, when walking with such a prosthesis, the full body weight of the patient is placed at least intermittently on the prosthesis. The pressure within the volume between the liner, pulled over the amputation stump, and the prosthesis system is also subject to considerable fluctuations. It is therefore possible for the pressure to lie briefly outside the range defined by the limit values without disadvantages occurring. Moreover, the optimal pressure in the volume is dependent on the state of movement of the prosthesis.

For example, if the patient is seated for some length of time, the pressure may be increased, such that the pressure difference between the pressure prevailing in the volume and the atmospheric pressure surrounding the prosthesis is reduced. Consequently, the holding force exerted on the prosthesis system by the negative pressure is also reduced. However, since the prosthesis is not loaded in this state, the patient does not feel it uncomfortable to wear. Further pumping of the vacuum is not necessary in this state.

To be able to differentiate between states of movement of this kind, it has been proposed, for example in WO 2008/073286 A1 and US 2011/0125291 A1, to integrate a computer into the prosthesis system, wherein certain characteristics in the chronological pressure profile of the individual states of movement are detected and, for each of the states of movement thus detected, separate limit values are set, between which the measured pressure should lie.

Although it is thereby possible to use pressure values that are adapted in each case for different states of movement, the set-up and the control method are complicated and therefore cost-intensive in particular for the patient. Moreover, the method is susceptible to error and requires considerable maintenance. Naturally, the individual pressure profiles vary from patient to patient in different states of movement, for example standing, sitting, running or walking, and it is therefore entirely possible that the computer fails to detect a concordance between the measured pressure profile and the stored comparison pressure profiles.

The object of the invention is therefore to propose a method for adjusting the pressure in the volume in a negative-pressure socket system, specifically a method that is easy to carry out, gives reliable and reproducible results and, in addition, provides the best possible pressure for the respective state of movement.

The invention achieves the stated object by means of a method of the type in question that comprises the following steps:

a) determining pressure values, which are a measure of the pressure in the volume, at different moments, and storing the pressure values that have been determined,
b) determining a maximum value and a minimum value from the stored pressure values,
c) determining a difference value from the maximum value and the minimum value,
d) comparing the difference value with a predetermined first limit value, and
e) activating a pump in order to aspirate a fluid from the volume if the difference value is greater than the predetermined first limit value.

Most states of movement of a leg prosthesis, for example walking or running, result in a periodically recurring pressure profile within the volume in the negative-pressure socket system. Thus, the pressure in the volume will increase when the prosthesis is loaded, such that the pressure difference between the pressure present in the volume and the atmospheric pressure surrounding the prosthesis system decreases. However, if the prosthesis is not loaded and the prosthetic foot is located in the swing phase, the pressure in the volume will drop, and the pressure difference between the pressure present in the volume and the atmospheric pressure surrounding the prosthesis system will increase. The invention is based on the concept that, independently of the state of movement, the difference between the maximum value and the minimum value of the pressure values thus detected should not exceed a predetermined first limit value.

This predetermined first limit value can surprisingly be seen as independent of the absolute value of the pressure prevailing in the volume. For the difference value, therefore, it is immaterial whether the patient is strolling about slowly or walking quickly. As long as the difference is less than the predetermined first limit value, it is ensured that the prosthesis has a secure hold on the amputation stump and the patient moreover has a comfortable and secure feel when wearing the prosthesis.

If, for example, the patient is sitting on a chair while wearing the prosthesis system, the difference value is very small. There is no periodically recurring pressure profile, such that the pressure prevailing in the volume is almost constant. Should the prosthesis system have a leakage through which, for example, atmospheric air can get into the volume in the negative-pressure socket system, the pressure in the volume will approximate more and more to the atmospheric pressure. However, since the prosthesis is unloaded, this is not problematic, at least in substantial pressure ranges, and it does not lead to an uncomfortable or unpleasant feel when wearing the prosthesis. Since the difference between the maximum value and the minimum value of the determined pressure values does not change in this procedure, the pump is not activated with the method according to the invention.

If the patient now gets up from the seated position and starts walking, there are periodically recurring pressure fluctuations, such that the difference between the maximum value and the minimum value of the stored pressure values greatly increases. The difference value may far exceed the predetermined first limit value, such that the pump is activated and fluid is aspirated from the volume. The pressure within the volume is thereby reduced, and the pressure difference with respect to the atmospheric pressure surrounding the prosthesis set-up increases.

However, the more the pressure within the volume is reduced, the smaller are the time fluctuations in the pressure profile within the volume. This means that the difference value between the maximum value and the minimum value of the stored pressure values drops if the pump is activated. Thus, depending on the state of movement of the prosthesis or of the patient wearing the prosthesis, the optimal pressure is adjusted without states of movement having to be detected, without pressure profile patterns having to be stored, or without different limit values having to be stored for different types of movement.

In a preferred embodiment, the pressure values are stored according to the first-in first-out principle, and only a predetermined maximum number of pressure values is stored. When this maximum number is reached, the oldest pressure value is deleted for a new pressure value that is to be stored. This corresponds to the aforementioned first-in first-out principle. It is thus ensured that the stored pressure values are in each case the updated pressure values and thus best reflect the current state of the prosthesis.

Advantageously, the minimum value, the maximum value and the difference value are determined at different moments, and method steps d) and e) are performed for each determined difference value. The maximum values, the minimum values and the difference values are determined at different moments that advantageously lie as close as possible to one another in time. For each difference value thus determined, the comparison with the predetermined first limit value advantageously also takes place, such that an activation of the pump can also take place if necessary for each determined difference value. It is thus ensured that changes in the pressure profile can be reacted to in good time, such that incorrect pressure adjustments in the volume can be quickly detected and rectified. Preferably at least 10, particularly preferably at least 50 or most preferably at least 100 difference values are determined per second.

Advantageously, the maximum value, the minimum value and the difference value are determined at regular time intervals, preferably after the storage of each pressure value. Regular determination of the difference value results in particularly efficient monitoring of the pressure within the volume. It is of course expedient for new pressure values to be determined and stored between two successive determinations of the respective difference value. Otherwise, two successive determinations of the difference value would give the same result. To ensure as far as possible seamless monitoring of the pressure within the volume and, at the same time, to permit a rapid reaction to fluctuations of the pressure value, it is advantageous, after each storage of a pressure value, to determine anew the corresponding maximum value and minimum value and, from these, the difference value, and to compare the latter with the predetermined first limit value and, if appropriate, activate the pump.

It has proven advantageous if the pump is deactivated when the difference value is less than the predetermined first limit value. It is thus ensured that the adjusted pressure within the volume is high enough to ensure a secure hold and a reliable function of the prosthesis on the amputation stump and, at the same time, low enough to avoid swelling, pressure sores and other kinds of discomfort when the patient is wearing the prosthesis.

If the patient moves with a leg prosthesis, the actual value of the difference value between the maximum value and the minimum value of the stored pressure values is in particular dependent on the pressure adjusted within the volume. The lower the pressure within the volume, i.e. the greater the difference between this pressure and the atmospheric pressure surrounding the prosthesis set-up or the negative-pressure socket system, the lower the difference value. In the previously described methods, the difference value was monitored merely to ascertain whether it is greater than the predetermined first limit value. Particularly in the event of the patient moving with the prosthesis, however, the difference value may also be too low. This is especially the case when the pressure within the volume has dropped to an extreme degree, which can in particular lead to deterioration in the wearing comfort. Therefore, in a preferred embodiment of the method, the pressure within the volume is increased if the difference value is less than a predetermined second limit value. The increase of the pressure within the volume can be brought about, for example, by now operating the pump in the opposite direction, such that it does not pump fluid out of the volume and instead pumps fluid into the volume. As an alternative to this, a valve provided for this purpose can also be opened.

In this case, however, the difference value is advantageously further monitored. In the moved state of the prosthesis, the difference value between the maximum value and the minimum value of the stored pressure values increases in this case. However, if the prosthesis is not in movement, for example because the patient is seated for a considerable period of time, the difference value remains very low, such that an increase of the pressure within the volume could, without further monitoring of the difference value, lead to the prosthesis detaching completely from the amputation stump. To avoid this, the pressure increasing the difference value is advantageously monitored, so as to be able to ascertain whether it changes when the pressure increases. If this is not the case, the increase of the pressure is ended.

Advantageously, the method additionally comprises the following steps:
f) calculating an average value from the stored pressure values,
g) establishing a comparison result by comparing the average value with a predetermined third limit value, and
h) activating the pump in accordance with the comparison result.

These method steps are expedient particularly in the case where the patient does not move the prosthesis for a considerable period of time, for example when seated. In this case, leakage can cause an increase of the pressure within the volume, such that the pressure difference between the pressure within the volume and the atmospheric pressure surrounding the prosthesis decreases. This can have the effect that, if the patient moves again after being seated, for example stands up or walks about, the negative pressure is too low, i.e. the pressure within the volume is too high, with the result that secure handling of the prosthesis may no longer be ensured. To avoid this, the average value of the stored pressure values is calculated and compared with a predetermined third limit value. The pump is controlled according to the comparison result thus determined.

The pressure values are preferably determined by establishing a difference between an atmospheric pressure surrounding the prosthesis system, or the negative-pressure socket system, and the pressure prevailing within the volume, and the pump is activated if the average value is less than the predetermined third limit value. If the pressure values are determined in this way, a leakage leads to an increase of the pressure in the interior of the volume in the negative-pressure socket system and therefore to a reduction of the corresponding pressure value. The pump must therefore be activated if the average value of the stored pressure values drops below the predetermined third limit value.

Alternatively to this, the pressure values can also be determined by detecting the pressure within the volume, and the pump can be activated if the average value is greater than the predetermined third limit value. In this case, absolute pressure sensors are used in order to determine the absolute pressure within the volume. A difference in relation to the atmospheric pressure surrounding the prosthesis would not be determined in this case. However, an increase of the pressure within the volume in this case also leads to an increase of the pressure values, such that the pump has to be activated if the average value is greater than the predetermined third limit value.

Independently of the method of measuring the individual pressure values, it has proven advantageous if the average value is a moving average value. This means, for example, that all of the stored pressure values are always used for the averaging.

As an alternative to the possibilities described above, the pump can also be deactivated if the average value thus determined has increased by a predetermined value. However, since the critical parameter for the wearing comfort and the safe use of the prosthesis is the difference value, the deactivation of the prosthesis is preferable to this possibility in the event that the difference value is less than the predetermined limit value. A combination of both criteria is of course also conceivable.

For reasons relating to its design, a vacuum pump arranged on a prosthesis system has a maximum pressure difference that it can produce between the volume in the negative-pressure socket system and the atmospheric pressure. Particularly in the case where the pressure values are determined as difference pressure value, the average value can therefore also be monitored to ascertain whether this maximum pressure predefined by the design of the pump is almost reached. If this is the case, and no other criterion leads to a switching-off of the pump, it would also be possible for the pump to be switched off for this reason alone in order to avoid an overloading of the pump. For design reasons, a further reduction of the pressure within the volume is not possible for the respective pump, such that a further reduction of the pressure cannot be achieved. In order to avoid an overloading of the pump, the latter should in this case be switched off.

Instead of a predetermined maximum pressure that is achievable as a result of the pump design, the change in the average value during the pumping operation could be considered. With the pump activated, the average value of the detected pressure should change perceptibly. If this is no longer the case, it is a clear indication that the pump has reached its performance limit and that further pumping has no appreciable effect. To save energy and to avoid subjecting the patient to any unnecessary noise, the pump should also be switched off in this case. All of these possibilities can be achieved by processing the determined average values and by using a corresponding control system, which can be arranged in the prosthesis system for example, and they can, if appropriate, be combined with one another.

An illustrative embodiment of the present invention is explained in more detail below with reference to a drawing in which:

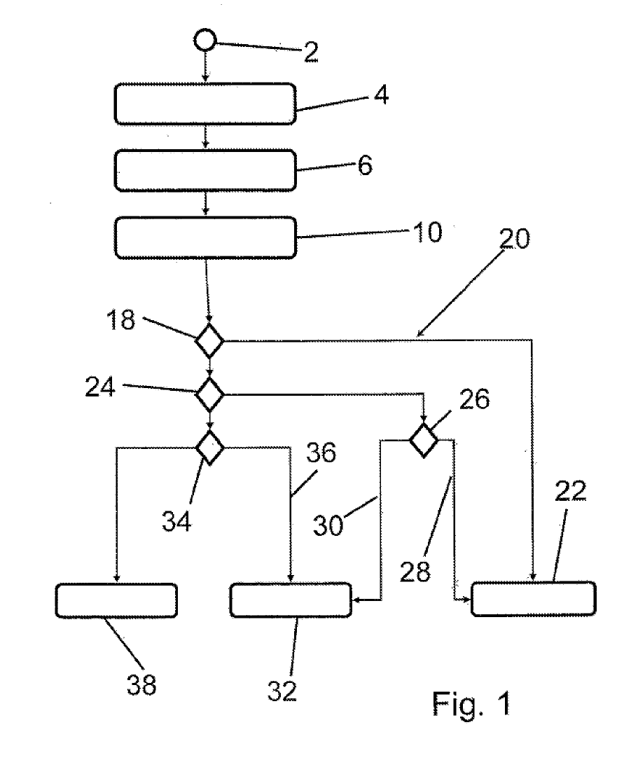

FIG. 1 shows a schematic flowchart of a method according to a first illustrative embodiment of the present invention,

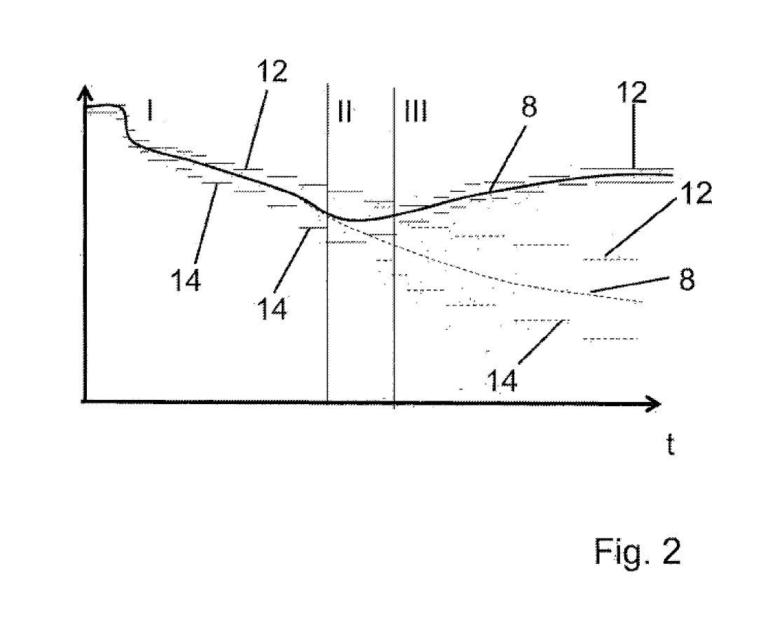

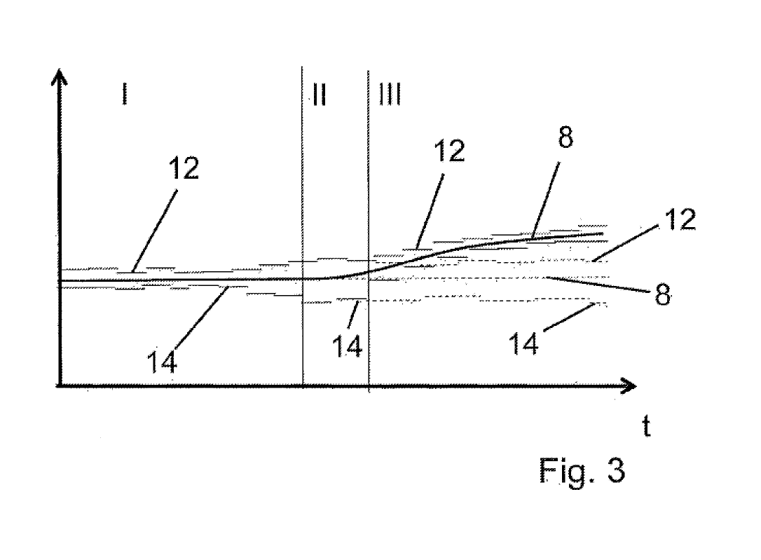

FIGS. 2 and 3 show the profiles of different measurement values during the implementation of the method according to a further illustrative embodiment of the present invention,

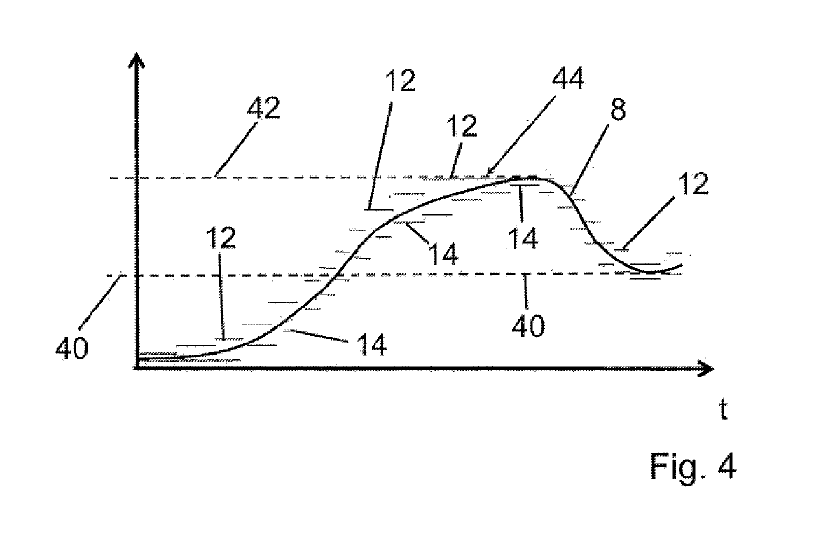

FIG. 4 shows a pressure profile from a control system according to the prior art in comparison with a control system according to an illustrative embodiment of the present invention,

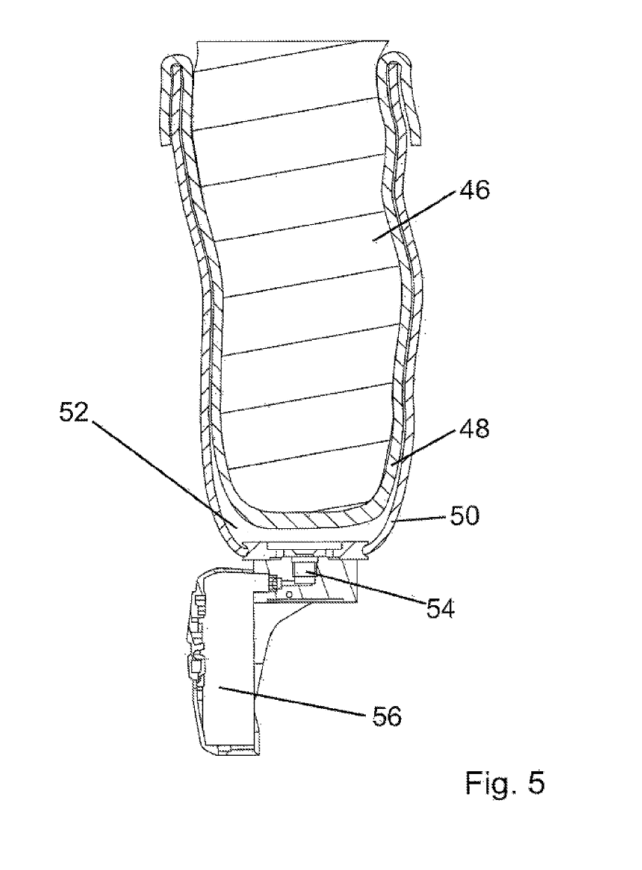

FIG. 5 shows the schematic cross-sectional view through a detail of a prosthesis system arranged on an amputation stump, and

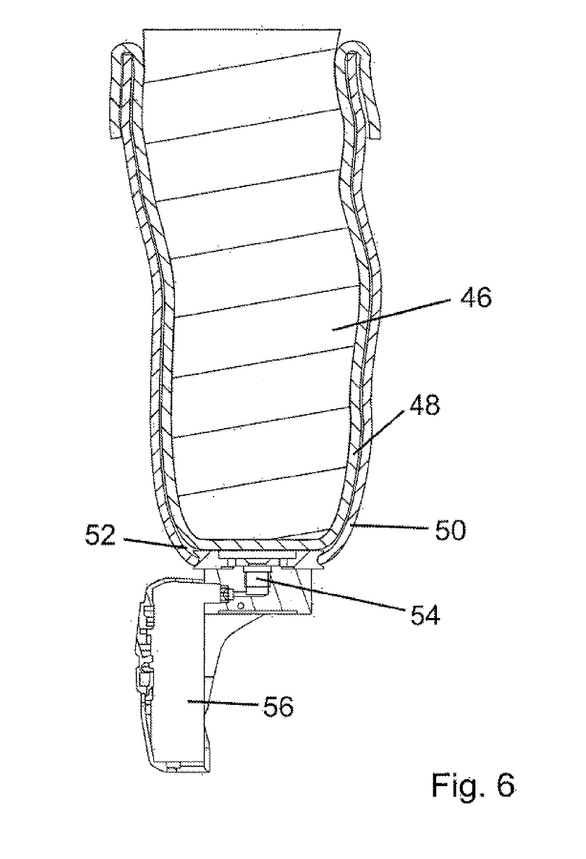

FIG. 6 shows the view from FIG. 5 after the negative pressure has been generated.

FIG. 1 shows the schematic sequence of a method according to a first illustrative embodiment of the present invention in the form of a flowchart. The method begins at a moment 2, which is used as a release moment, timer event or trigger. This is followed by the determination of the respective pressure value 4, which is a measure of the pressure in the volume. This can be done, for example, by an absolute pressure sensor or a differential pressure sensor, wherein a differential pressure sensor determines the pressure difference between an atmospheric pressure surrounding the prosthesis and the pressure in the volume. The pressure value thus determined is additionally stored in a memory element. If a predetermined maximum number of pressure values is already stored in the memory element, the oldest pressure value is preferably removed from the memory. This corresponds to what is known as the first-in first-out principle, which is preferably used.

Averaging 6 is then carried out to determine an average value 8 from all the stored pressure values. This can be an arithmetic mean or a weighted average that takes account of certain circumstances. Weighted averages are in particular advantageously used if the individual pressure values were not recorded at measurement points equidistant in time. The formation of a weighted average can take account of the time intervals of different lengths between the individual pressure values. A difference value 16 is thus determined from the difference of the maximum value 12 and the minimum value 14. In the subsequent course of the method, the average value 8 and the difference value 16 are used to control the pump, which is responsible for adjusting the pressure in the volume.

For this purpose, in a first comparison 18 in the illustrative embodiment shown in FIG. 1, the average value 8 is compared with a predetermined third limit value. If the pressure values were recorded by differential pressure sensors, a reduction of the pressure value signifies a rise of the pressure within the volume, since the difference between the pressure in the volume and the atmospheric pressure surrounding the prosthesis drops. If the difference becomes too small, the pump has to be activated so as to be able to produce a sufficiently strong negative pressure in the volume between the liner, pulled over the amputation stump, and the prosthesis system.

Therefore, in the first comparison 18, the average value 8 is compared with the predetermined third limit value. If the average value 8 in the illustrative embodiment shown is less than the third limit value, this leads to a pump activation 22, as is indicated by a first direction arrow 20. If the average value 8 is greater than the predetermined third limit value, the difference value 16 is compared with the predetermined first limit value in a second comparison 24. If the difference value 16 is greater than the predetermined first limit value, a check 26 is made to ascertain whether the average value 8 has already reached the limit value permitted for the pump by the design. If this is not so, the arrow 28 likewise leads to the pump activation 22. In this way, the pressure in the volume between liner and prosthesis element is reduced, which has the effect that, since these are differential pressure measurements, the average value 8 from the stored pressure values increases. This results in a reduction of the difference value 16.

By contrast, if the average value 8 is already at the performance maximum of the pump in question, a further increase of the average value 8, with an associated drop in the pressure within the volume, is then no longer possible with the pump used, and therefore the method follows the arrow 30, with the effect that the pump is not activated, as is indicated by the box 32.

If, in the second comparison 24 in which the difference value 16 is compared with the predetermined first limit value, it is found that the difference value 16 is less than the predetermined first limit value, a check 34 is made in order to ascertain whether the maximum value 12 corresponds to the minimum value 14. If this is not so, the arrow 36 leads to the box 32, and therefore the pump is not activated. However, if the minimum value 14 corresponds to the maximum value 12, the difference value is almost zero within the context of the measurement accuracy. A pressure increase 38 can then take place, as a result of which the pressure in the volume increases and, therefore, the average value 8 is lowered. With this method, an optimal pressure is obtained in the volume in the negative-pressure socket system in any state of movement of the prosthesis.

In FIGS. 2 and 3, the average value 8 is shown as a function of the time t, in each case in the form of a solid line. The thin solid strokes above the respective average value 8 show the maximum value, while those below the average value 8 show the minimum value 14. An increase of the average value 8 signifies in each case a stronger negative pressure in the volume in the negative-pressure socket system and, therefore, a firm hold of the prosthesis.

It will be seen that the diagrams in FIGS. 2 and 3 are divided into three areas I, II and III.

In the areas I and III, the difference between the minimum value 14 and the maximum value 12, which difference corresponds to the difference value 16, is in each case less than the predetermined first limit value, such that no pump activation takes place in these areas. In the area II in FIGS. 2 and 3, the difference value 16 is greater than the predetermined first limit value, such that an activation of the pump takes place in each case in the area II. This results in an increase of the average value 8 and a reduction of the difference value 16 at longer times t.

However, in the illustrative embodiments of the present invention that are shown in FIGS. 2 and 3, the pump is not deactivated as soon as the difference value 16 is less than the predetermined limit value. If this were the case, the average value 8 would not be permitted to increase further in the respective area III. Here, another criterion has been chosen for the deactivation of the pump.

It will be seen particularly in FIG. 2 that, in the area I, there is in part a marked decrease in the average value 8. This can be the result of leakage, for example, through which a fluid can penetrate into the volume. This has the effect of reducing the average value 8, from which it can already be seen that the average value and therefore also the pressure values are in the form of differential pressure values, which represent the difference between the atmospheric pressure surrounding the prosthesis and the pressure in the volume. A reduction of this average value 8 therefore signifies an increase of the pressure in the volume.

In FIG. 2 and also in FIG. 3, broken lines indicate how the average value 8 and also the minimum value 14 and maximum value 12 would develop in the absence of a pump activation. It will be seen that the average value 8 drops more or less steeply in both figures, and the respective difference value 16, as the difference between the maximum value 12 and the minimum value 14, sharply increases according to the time t. This would mean that the prosthesis felt uncomfortable to wear and insecure, since the patient would have the feeling that the prosthesis was loose, unstable and not able to be loaded with any confidence.

The present method provides a pressure control for the volume between the liner and the prosthesis element, which control requires only a small number of measurement values, functions in a safely reproducible manner and, in addition, is simple in terms of design and control technology.

FIG. 4 also shows the average value 8 as a function of time t. However, the average value 8 in the profile shown in FIG. 4 corresponds to a pressure that is controlled by a control system according to the prior art. Here, a pump is controlled such that the average value 8 always lies between a previously fixed lowest value 40 and a previously fixed highest value 42. In the left-hand area, the average value 8 is well below the lowest value 40, such that a pump is activated. A fluid is thereby removed from the volume, such that a negative pressure is established, as can be identified from a clear increase of the average value 8. The pump is deactivated only when the average value 8 reaches the highest value 42. The average value 8 then drops off, which can be caused by leakages for example. However, these are shown in greatly exaggerated form in FIG. 4. The reduction of the negative pressure, associated with the reduction of the average value 8, can in reality extend to several hours and depends among other things on the state of loading of the prosthesis.

As soon as the average value 8 in the right-hand area of FIG. 4 again reaches the predetermined lowest value 40, the pump is activated again, such that the average value 8 increases again.

In FIG. 4, as in FIGS. 2 and 3, the maximum values 12 and the minimum values 14 are also shown. It will be noted that the difference value 16 between maximum value 12 and minimum value 14 is quite considerable, particularly in the left-hand area of FIG. 4 where the average value 8 increases, such that, using a method according to an illustrative embodiment of the present invention, the pump would also have been activated in order to obtain a stronger negative pressure in the volume in the negative-pressure socket system. However, particularly in an area 44 located shortly before the point where the average value 8 reaches the predetermined highest value 42, the difference value 16 between the maximum value 12 and the minimum value 14 is quite small, such that a control method according to an illustrative embodiment of the present invention would already have switched the pump off in this area. As a result, the noise the patient would have heard from the pump would have been less noticeable and in particular would have lasted a shorter period of time. Moreover, the negative pressure generated in the volume would not have been so great, but it would nonetheless have been sufficient for the state of loading of the prosthesis.

In the area where the average value 8 drops off in FIG. 4, it can clearly be seen that the difference value 16 between the maximum value 12 and the minimum value 14 increases considerably. In this area, the pump would have been activated again by a method according to an illustrative embodiment of the present invention, with the result that such a marked decrease of the negative pressure as shown in FIG. 4 would have been avoided. For the patient, this would mean far fewer fluctuations in the adjusted negative pressure and, therefore, also far fewer fluctuations in the forces acting on the amputation stump. The wearing comfort of the prosthesis is greatly increased in this way.

FIG. 5 shows a detail of a schematic cross-sectional view. It depicts an amputation stump 46, over which a liner 48 has been pulled. The liner 48 is arranged in a prosthesis socket 50, and a volume 52 is located between the liner 48 and the prosthesis socket 50.

This volume 52 is connected via a fluid connection 54 to a pump 56, by means of which a negative pressure can be produced in the volume 52. This is shown in FIG. 6. After the pump 56 is activated, a negative pressure is produced in the volume 52, as a result of which the amputation stump 46 and the liner 48 pulled over the latter are drawn into the prosthesis socket 50 and adapt to the shape of the prosthesis socket. It will be seen that the volume 52 still has a minimal size. The liner 48, with the amputation stump 46 located therein, is held in the prosthesis socket 50 by the negative pressure that is present in this volume 52.

### LIST OF REFERENCE SIGNS

I, II, III area
t time
2 moment
4 determining the pressure value
6 averaging
8 average value
10 evaluation of extremes
12 maximum value
14 minimum value
16 difference value
18 first comparison
20 direction arrow
22 pump activation
24 second comparison
26 check
28 arrow
30 arrow
32 box
34 check
36 arrow
38 pressure increase
40 lowest value
42 highest value
44 area
46 amputation stump
48 liner
50 prosthesis socket
52 volume
54 fluid connection
56 pump

1. A method for adjusting a pressure in a volume in a negative-pressure socket system, said method comprising the following steps:
a) determining pressure values, which are a measure of the pressure in the volume, at different moments, and storing the pressure values that have been determined;
b) determining a maximum value and a minimum value from the stored pressure values;
c) determining a difference value from the maximum value and the minimum value;
d) comparing the difference value with a predetermined first limit value;
e) activating a pump in order to aspirate a fluid from the volume if the difference value is greater than the predetermined first limit value.

2. The method as claimed in claim 1, wherein the pressure values are stored according to a first-in first-out principle, and only a predetermined maximum number of pressure values is stored.

3. The method as claimed in claim 1, wherein the maximum value, the minimum value and the difference value are determined at different moments, and method steps d) and e) are performed for each determined difference value.

4. The method as claimed in claim 3, wherein the maximum value, the minimum value and the difference value are determined at regular time intervals.

5. The method as claimed in claim 1, wherein the pump is deactivated if the difference value is less than the predetermined first limit value.

6. The method as claimed in claim 1, wherein the pressure in the volume is increased if the difference value is less than a predetermined second limit value.

7. The method as claimed in claim 1, and including the additional steps of:
f) calculating an average value from the stored pressure values;
g) establishing a comparison result by comparing the average value with a predetermined third limit value, and
h) activating the pump in accordance with the comparison result.

8. The method as claimed in claim 7, wherein the pressure values are determined by establishing a difference between an atmospheric pressure surrounding the negative-pressure socket system and the pressure prevailing in the volume, and the pump is activated if the average value is less than the predetermined third limit value.

9. The method as claimed in claim 7, wherein the pressure values are determined by detecting the pressure in the volume, and the pump is activated if the average value is greater than the predetermined third limit value.

10. The method as claimed in claim 7, wherein the average value is a moving average.

11. The method as claimed in claim 4, wherein the maximum value, the minimum value and the difference value are determined after the storage of each pressure value.

12. A method for adjusting a pressure in a volume of a prosthetic socket, the method comprising:
determining pressure values in the volume at different moments;
determining a maximum value and a minimum value from the pressure values;
determining a difference value between the maximum value and the minimum value;
comparing the difference value with a predetermined first limit value;
aspirating a fluid from the volume if the difference value is greater than the predetermined first limit value.

13. The method as claimed in claim 12, further comprising storing the pressure values according to a first-in first-out principle, and only a predetermined maximum number of pressure values are stored.

14. The method as claimed in claim 12, wherein the maximum value, the minimum value, and the difference value are determined at different moments.

15. The method as claimed in claim 14, wherein the maximum value, the minimum value, and the difference value are determined at regular time intervals.

16. The method as claimed in claim 12, further comprising ceasing aspirating the fluid if the difference value is less than the predetermined first limit value.

17. The method as claimed in claim 12, further comprising increasing pressure in the volume if the difference value is less than a predetermined second limit value.

18. The method as claimed in claim 12, further comprising:
calculating an average value from the stored pressure values;
establishing a comparison result by comparing the average value with a predetermined third limit value;
aspirating the fluid in accordance with the comparison result.

19. The method as claimed in claim 18, wherein the pressure values are determined by establishing a difference between an atmospheric pressure and a current pressure value in the volume, and the aspirating occurs if the average value is less than the predetermined third limit value.

20. The method as claimed in claim 18, wherein the pressure values are determined by detecting a current pressure valvue in the volume, and aspirating occurs if the average value is greater than the predetermined third limit value.
